(19) United States
(12) Patent Application Publication
(10) Pub. No.: US 2015/0165414 A1
(43) Pub. Date: Jun. 18, 2015

(54) METHODS AND REACTORS FOR PRODUCING ACETYLENE

(71) Applicant: UOP LLC, Des Plaines, IL (US)

(72) Inventors: Rajeswar R. Gattupalli, Arlington Heights, IL (US); Laura E. Leonard, Carol Stream, IL (US)

(73) Assignee: UOP LLC, Des Plaines, IL (US)

(21) Appl. No.: 14/169,069

(22) Filed: Jan. 30, 2014

Related U.S. Application Data
(63) Continuation-in-part of application No. 14/104,728, filed on Dec. 12, 2013.

(51) Int. Cl. B01J 19/10; C07C 2/78

(52) U.S. Cl. CPC B01J 19/10; C07C 2/78

(57) ABSTRACT

Methods and reactors are provided for producing acetylene. The method includes combusting a fuel with oxygen in a combustor to produce a carrier gas, and accelerating the carrier gas to a supersonic speed in a converging/diverging nozzle prior to the carrier gas entering a reaction zone. A nozzle exit temperature of the carrier gas is controlled from about 1,200° C. to about 2,500° C. Methane is added to the carrier gas in the reaction zone, and kinetic energy in the carrier gas is converted to thermal energy in the reaction zone to increase the temperature of the carrier gas such that the methane reacts by pyrolysis to form the acetylene.

This application is a Continuation-in-Part (CIP) of U.S. application Ser. No. 14/104,728, entitled Methods and Reactors for Producing Acetylene, filed Dec. 12, 2013.

### TECHNICAL FIELD

The present disclosure generally relates to methods and reactors for producing hydrocarbons, and more particularly relates to methods and reactors for producing acetylene using a pyrolysis reaction.

### BACKGROUND

Light olefin materials, including ethylene, represent a large portion of the worldwide demand in the petrochemical industry. Ethylene is used in the production of numerous chemical products via polymerization, oligomerization, alkylation, and other well-known chemical reactions. As such, ethylene is an essential building block for the modern petrochemical and chemical industries. Producing large quantities of ethylene in an economical manner, therefore, is a focus of the petrochemical industry. Presently, the main source for ethylene is from cracking petroleum feeds. However, due at least in part to the large demand for ethylene and other light olefinic materials, the cost of appropriate petroleum feeds has steadily increased.

Natural gas includes large quantities of methane, and the cost of natural gas has fallen while costs for traditional petroleum feeds have increased. However, efforts to convert natural gas to ethylene by pyrolysis have not produced an economically viable option. Methane has been converted to acetylene in some pyrolysis reactors, and the acetylene can then be hydrogenated to form ethylene. Control of the temperature at various locations in the pyrolysis reactor is needed for high yields of acetylene, and to limit unwanted byproducts such as soot. Many pyrolysis reactions are run at very high temperatures, and adequate temperature control has not been demonstrated.

Accordingly, it is desirable to develop methods and apparatuses for controlling the temperature of a pyrolysis reaction when converting a methane feed to acetylene. In addition, it is desirable to develop methods and apparatuses for controlling reaction temperatures to prevent pyrolysis until desired, and then to initiate and control the pyrolysis reaction to increase yields of acetylene. Furthermore, other desirable features and characteristics of the present embodiment will become apparent from the subsequent detailed description and the appended claims, taken in conjunction with the accompanying drawings and this background.

### BRIEF SUMMARY

Methods and reactors for producing acetylene are provided. In an exemplary embodiment, a method includes combusting a fuel with oxygen in a combustor to produce a carrier gas, and accelerating the carrier gas to a supersonic speed in a converging/diverging nozzle prior to the carrier gas entering a reaction zone. A nozzle exit temperature of the carrier gas is controlled from about 1,200° C. to about 2,500° C. Methane is added to the carrier gas in the reaction zone, and kinetic energy in the carrier gas is converted to thermal energy in the reaction zone to increase the temperature of the carrier gas such that the methane reacts by pyrolysis to form the acetylene.

In some embodiments, the nozzle exit temperature is controlled by adding a heat sink gas to the carrier gas before the reaction zone. In some embodiments, one or more of the fuel, the oxygen, and the heat sink gas can be preheated.

In accordance with a further exemplary embodiment, a reactor for producing acetylene is provided. The reactor includes a combustor with a fuel inlet and an oxygen supply inlet. The combustor is fluidly coupled to a converging/diverging nozzle that is configured to accelerate a carrier gas to supersonic speeds. The converging/diverging nozzle is fluidly coupled to a reaction zone that includes a methane inlet. There is a nozzle heat sink gas inlet in the reactor between the combustor and the reaction zone.

### BRIEF DESCRIPTION OF THE DRAWING

The present embodiment will hereinafter be described in conjunction with the following drawing FIGURES, wherein like numerals denote like elements, and wherein:

The FIGURE is a schematic diagram of an exemplary embodiment of an apparatus and a method for producing acetylene.

### DETAILED DESCRIPTION

The following detailed description is merely exemplary in nature and is not intended to limit the application and uses of the embodiment described. Furthermore, there is no intention to be bound by any theory presented in the preceding background or the following detailed description.

The various embodiments described herein relate to methods and reactors for producing acetylene from methane by a pyrolysis reaction. The process is designed to provide sufficient enthalpy and heat for the pyrolysis reaction. The enthalpy can be increased in a number of ways. One or both of the fuel and oxygen can be preheated. Superheated steam can be used alone, or in combination with preheating one or both of the fuel and oxygen. The system can be run with excess fuel, which can be used alone, or in combination with preheating one or both of the fuel and oxygen. The conversion and selectivity of the reaction change depending on the enthalpy of the carrier gas.

A fuel is burned with oxygen in a combustor, and the combustion gases are accelerated to supersonic speed in a converging/diverging nozzle. The combustion gases serve as a carrier gas, and the acceleration of the carrier gas converts thermal energy to kinetic energy to lower the temperature of the carrier gas below the temperature needed for pyrolysis. Methane is injected into the carrier gas in a reaction zone while the carrier gas is flowing at supersonic speeds. Back pressure is used in the reaction zone to create a shock wave that converts the kinetic energy of the carrier gas back into thermal energy to increase the temperature and cause the methane to react by pyrolysis. A quench is then used to prevent the pyrolysis reaction from continuing, so the reaction is terminated after acetylene is formed but before significant quantities of the acetylene react to form soot or hydrocarbons with 3 or more carbon atoms. The acetylene yield from the pyrolysis reaction is optimized when the carrier gas temperature entering the reaction zone is controlled from about 1,200° C. to about 2,500° C., or from about 1,500° C. to about 1,900° C.

The temperature of the supersonic carrier gas can be controlled by adding a heat sink gas to the carrier gas upstream from the reaction zone. Several different heat sink gases can be used, and various heat sink gas injections points are possible. The heat sink gas is heated by the carrier gas such that the mixture is within the desired temperature range when entering the reaction zone.

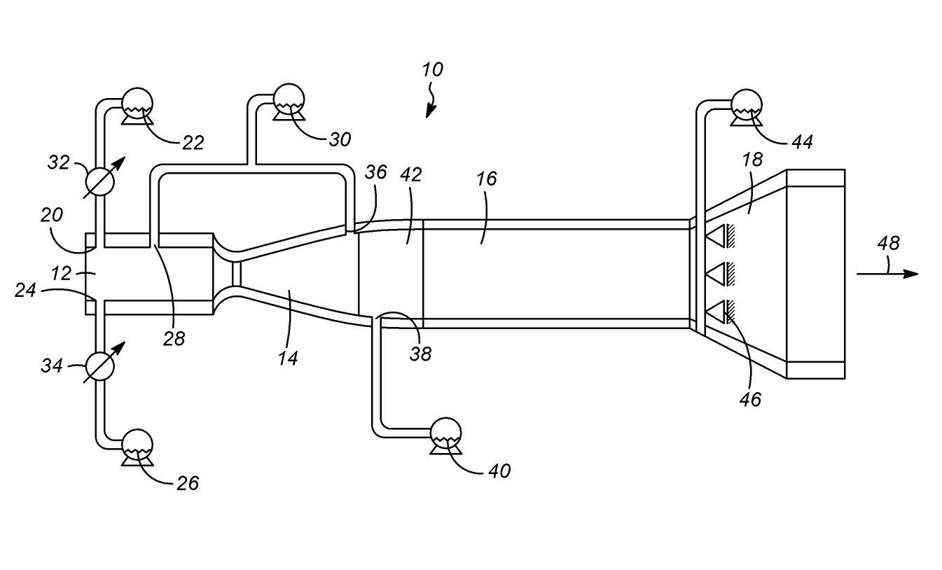

Reference is now made to FIG. 1. A reactor 10 includes a combustor 12 fluidly coupled to a converging/diverging nozzle 14, and a reaction zone 16 fluidly coupled to the converging/diverging nozzle 14. A quench zone 18 is fluidly coupled to reaction zone 16 such that fluid flows from the combustor 12 through the converging/diverging nozzle 14, through the reaction zone 16, and then through the quench zone 18. The reactor 10 is shown as a single vessel, but it should be understood that the reactor 10 may be formed modularly or as separate vessels. The modules or separate components of the reactor 10 may be joined together permanently or temporarily, or may be separate from one another with fluids contained therein by other means, including but not limited to differential pressure.

The combustor 12 includes a fuel inlet 20 that provides a fuel 22, and an oxygen supply inlet 24 that provides oxygen from an oxygen supply 26. The fuel 22 may be a wide variety of compounds that can be burned or oxidized, including but not limited to hydrogen, methane, or other hydrocarbons. The oxygen may be relatively pure, such as about 90 mass percent oxygen or greater, but in other embodiments the oxygen supply 26 can be other oxygen-containing streams with lower oxygen concentrations. One non-limiting example of oxygen supply 26 includes air, which is about 21 percent oxygen and about 78 percent nitrogen when dry. The fuel inlet 20 and the oxygen supply inlet 24 include injectors, nozzles, open ports, or other means for introducing the fuel 22 and the oxygen supply 26 into the combustor 12. The fuel inlet 20 and oxygen supply inlet 24 can be introduced into the combustor 12 in a wide variety of manners, including an axial direction, tangential direction, radial direction, other directions, or a combination thereof. In some embodiments, the combustor 12 also includes a combustor heat sink gas inlet 28 for introducing a heat sink gas 30 into the combustor. The use and operation of the heat sink gas 30 is described more fully below.

The fuel 22 and oxygen ignite and burn in the combustor 12, and the combustion gases formed serve as a carrier gas within the reactor 10. In some embodiments, the fuel, the oxygen, or both are preheated, while in others they are not preheated. In an exemplary embodiment, the fuel 22 is about 95 mass percent or more hydrogen that is preheated to about 800° C., and the oxygen supply 26 includes about 90 mass percent or more oxygen. A fuel heater 32 can be used to heat the fuel 22 before entering the combustor 12, and an oxygen supply heater 34 can be used to heat the oxygen supply 26 before entering the combustor 12. In some embodiments, the fuel 22 and oxygen supply 26 are heated to provide sufficient enthalpy for the pyrolysis reaction. The temperature of the carrier gas generated by combusting the fuel 22 and oxygen is about 3,200° C. to about 3,300° C. in one embodiment, and the carrier gas flows out of the combustor 12 to the converging/diverging nozzle 14. In an alternate embodiment, the fuel 22 is about 95 mass percent or more methane, and in yet other embodiments the fuel 22 is a mixture of hydrogen and methane, or other hydrocarbons. In many embodiments, the fuel 22 and oxygen supply 26 are heated sufficiently to produce carrier gas exiting the combustor 12 at a temperature of about 2,500° C. to about 3,500° C.

The carrier gas is accelerated to supersonic speeds in the converging/diverging nozzle 14, where the converging/diverging nozzle 14 serves as a supersonic expander. The converging/diverging nozzle comprises a converging section fluidly coupled to a diverging section. The cross sectional area of the converging section decreases from the inlet to the outlet. The cross sectional area of the diverging section increases from the inlet to the outlet. The pressure in the combustor 12 is higher than in the reaction zone 16, so the carrier gas flows from the combustor 12 to the reaction zone 16. The carrier gas velocity will increase in the converging section of the converging/diverging nozzle 14 up to a maximum of Mach 1 at the throat. The carrier gas then further accelerates in the diverging section of the converging/diverging nozzle 14 as long as the pressure difference between the combustor 12 and the reaction zone 16 is sufficient. In some embodiments, a natural shock wave is generated at a point either within the converging/diverging nozzle 14 or near the exit of the converging/diverging nozzle 14, where the carrier gas flow rate drops from a speed of Mach 1 or higher to below supersonic speeds at the shock wave. The position of the shock wave can be moved, or the shock wave can be eliminated, by adjusting the pressure difference between the combustor 12 and the back pressure in the reaction zone 16.

Accelerating the carrier gas converts some of the thermal energy of the carrier gas to kinetic energy, so the temperature of the carrier gas lowers as it is accelerated. In an exemplary embodiment, the carrier gas is accelerated to a speed of about Mach 2 to about Mach 4, and in another embodiment the carrier gas is accelerated to a speed of about Mach 2.5 to about Mach 3.5, but other speeds are possible. A larger acceleration of the carrier gas requires higher pressures in the combustor 12, including higher pressures in the fuel 22 and oxygen supply 26 feed lines to the combustor 12. The higher pressures require higher pressure ratings for the associated equipment, and also result in higher operating costs to pressurize the feed streams. In an exemplary embodiment under adiabatic conditions with a stoichiometric mix of oxygen and hydrogen fuel 22, wherein the oxygen supply 26 is about 90 mass percent oxygen or higher, the temperature of the carrier gas exiting the converging/diverging nozzle 14 (referred to herein as the nozzle exit temperature) at about Mach 3 is about 2,300° C. A stoichiometric mix of oxygen and fuel 22 means a mixture where all the fuel 22 and all the oxygen in the oxygen supply 26 react together during combustion. Increasing the acceleration to a higher Mach speed further lowers the nozzle exit temperature, while increasing the pressure in the combustor 12 and lowering the acceleration does the opposite.

The converging/diverging nozzle 14 optionally includes a nozzle heat sink gas inlet 36. In some embodiments, the nozzle heat sink gas inlet 36 is positioned at the beginning of the converging/diverging nozzle 14, which is essentially at the outlet of the combustor 12. In alternate embodiments, the nozzle heat sink gas inlet 36 is positioned in the converging section of the converging/diverging nozzle 14, or the diverging section, or even in an optional straight section that may positioned before, between, or after the converging and diverging sections. The converging/diverging nozzle 14 may include the straight section (not shown) that does not converge or diverge, where the straight section can allow for mixing, temperature equilibration, or stabilization of the carrier fluid gas flow prior to entering the reaction zone 16.

The reaction zone 16 receives the carrier fluid from the converging/diverging nozzle 14 at supersonic speeds. A methane inlet 38 is positioned within the reaction zone 16 at or near its beginning, and methane gas from a methane supply 40 is injected into the reaction zone 16 through the methane inlet 38. The methane inlet 38 may include one or more injectors, nozzles, or other openings for introducing the methane supply 40 to the reaction zone 16. The reaction zone 16 may include a mixing zone 42 extending from the about the methane inlet 38 to a position further downstream of the methane inlet 38. The mixing zone 42, if present, is an area where the methane is allowed to mix with the carrier gas upstream from any shock waves introduced into the reaction zone 16, as described more fully below. The methane inlet 38 may introduce the methane gas axially, radially, tangentially, or other directions, or any combination thereof. The methane accelerates to supersonic speeds as it is mixed with the carrier gas.

The methane supply 40 may include other components in various embodiments. In some embodiments, the methane supply 40 is natural gas provided from a wide variety of sources, including but not limited to gas fields, oil fields, coal fields, fracking of shale fields, biomass, and landfill gas. In other embodiments, the methane supply 40 may be provided from an oil refinery or processing plant. For example, light alkanes, including methane, are often separated during processing of crude oil into various products, and the methane supply 40 may be provided from one of these sources. The methane supply 40 may also be provided by a variety of different sources, which are mixed or sequentially used, and the source of the methane supply 40 may be local or remote. In one embodiment, the methane supply 40 includes about 65 to about 100 mole percent methane. In another embodiment, the methane supply 40 includes about 80 to about 100 mole percent methane, and in yet another embodiment the methane supply 40 includes about 90 to about 100 mole percent methane. The remainder of the methane supply 40 may include many other compounds, such as ethane, propane, aromatics, other hydrocarbons such as aromatics, paraffins, or olefins, and other impurities such as sulfur containing compounds.

A shock wave is formed in the reaction zone 16 and converts some of the kinetic energy of the carrier gas and methane to thermal energy. The thermal energy increases the temperature of the carrier gas and methane to induce an endothermic pyrolysis reaction. The shock wave can be formed by back pressure, where the back pressure can be created in several different ways. For example, a flow restriction can be used to create a standing shock wave in the reaction zone 16, or pressurized gas can be injected into the reactor 10 to create a pulsed or standing shock wave. The carrier gas is quenched with a quench fluid 44 in the quench zone 18 to stop the pyrolysis reaction, and thereby reduce or prevent the production of larger molecules with more than 2 carbon atoms. The temperature of the quench fluid 44 is below the temperature of the pyrolysis reaction, and many different types of quench fluids 44 can be used. In an exemplary embodiment, the pyrolysis reaction is quenched with water injected through a quench fluid inlet 46, such as spray nozzles, injectors, or other devices. Steam or water is relatively easy to separate from the acetylene or other hydrocarbons produced, but other quench fluids 44 can also be used.

The mixed carrier gas and methane are below the pyrolysis reaction temperature before the shock wave, so the pyrolysis reaction does not begin until the carrier gas and methane enter the shock wave. Methane may begin the pyrolysis reaction at temperatures greater than about 1,500° C., and the rate of the pyrolysis reaction increases as the temperature increases. The temperature of the carrier gas should be low enough that the rate of the pyrolysis reaction is slow, yet have enough kinetic energy that the temperature can be increased to induce a rapid pyrolysis reaction when desired. The temperature of the carrier gas at the exit of the converging/diverging nozzle 14 is referred to as the nozzle exit temperature, as described above. The nozzle exit temperature is controlled from about 1,500° C. to about 1,600° C. in one embodiment, and from about 1,500° C. to about 1,900° C. in another embodiment. In yet another embodiment, the nozzle exit temperature is controlled from about 1,200° C. to about 2,500° C. Temperature control at the nozzle exit prevents or limits the pyrolysis reaction before the mixed carrier gas and methane reach the shock wave, which produces a more controlled reaction with higher yields of acetylene and less soot, other hydrocarbons with more than 2 carbon atoms, and carbon monoxide. As such, acetylene yields are maximized by controlling the temperature as described above. The acetylene and other products of the pyrolysis reaction, as well as the carrier gas, any unreacted methane, and any other components in the reactor 10 are discharged in a reactor discharge stream 48. The reactor discharge stream 48 has a higher concentration of acetylene than any of the inlet streams, including the methane supply 40. The acetylene in the reactor discharge stream 48 can be used or further processed in a variety of manners, including but not limited to direct use as a fuel or hydrogenation to from ethylene.

A heat sink gas 30 is introduced to the reactor 10 to control the nozzle exit temperature as described above. The temperature of the heat sink gas 30 is lower than the temperature of the carrier gas at the point where the heat sink gas 30 is added to the carrier gas, so some of the heat from the carrier gas is transferred to the heat sink gas 30. In many embodiments, the heat sink gas 30 is non-reactive or has a low reactivity at the pyrolysis reaction conditions. In some embodiments, water in the form of steam is added to the carrier gas as the heat sink gas 30. Water is a combustion gas, so it does not introduce any new chemical components into the reactor 10. Alternate chemicals that can be used as the heat sink gas 30 include carbon monoxide, carbon dioxide, nitrogen, or any of the noble gases such as neon, helium, or argon. In some embodiments, carbon monoxide and carbon dioxide are present as combustion gases from the combustor, so these compounds are naturally present in the reaction zone 16. The potential heat sink gas compounds listed above have a relatively low reactivity at the pyrolysis reaction conditions, and are compounds other than those primarily present in the fuel 22 or the oxygen supply 26. Other possible heat sink gases 30 include excess hydrogen, methane, or other fuels 22. The fuel 22, which may include hydrogen and/or methane, can be added in excess of the stoichiometric oxygen to fuel ratio, where the excess fuel 22 serves as a heat sink gas 30. It is also possible to use other compounds as the heat sink gas 30 in alternate embodiments. The heat sink gas can be preheated in some embodiments, if desired. For example, superheated steam can be used as a heat sink gas. When excess fuel is used as a heat sink, the fuel can be preheated.

In some embodiments, the heat sink gas 30 is added to the reactor 10 through the combustor heat sink gas inlet 28 (if present) and/or the nozzle heat sink gas inlet 36, if present. The nozzle heat sink gas inlet 36 is positioned in one or more locations in the converging/diverging nozzle 14, so the nozzle heat sink gas inlet 36 is between the combustor 12 and the reaction zone 16. In an exemplary embodiment, the nozzle heat sink gas inlet 36 is positioned at or near the exit of the converging/diverging nozzle 14. The converging/diverging nozzle 14 is exposed to high velocity, varying pressures, and high temperatures, which makes it a relatively severe location, so positioning the nozzle heat sink gas inlet 36 at the nozzle exit minimizes additional stress. However, in other embodiments, the nozzle heat sink gas inlet 36 is positioned in the converging or diverging sections of the converging/diverging nozzle 14, or even at the throat, which provides better mixing of the heat sink gas 30 and the carrier gas.

Adding the heat sink gas 30 into the combustor 12 provides good mixing and temperature equilibration between the heat sink gas 30 and the carrier gas, but the combustor 12 operates at higher pressures than the converging/diverging nozzle 14 and the reaction zone 16. Therefore, higher pressures are needed to introduce the heat sink gas 30 into the combustor 12 than into the converging/diverging nozzle 14. Less pressure is needed to add the heat sink gas 30 into the converging/diverging nozzle 14, and lower pressures can reduce energy costs for pressurization and capital costs for equipment with higher pressure ratings.

In some embodiments, excess hydrogen, methane, or other types of fuel 22 are added to the combustor 12 through the fuel inlet 20 or through combustor heat sink gas inlet 28 in greater than the stoichiometric oxygen to fuel ratio such that the excess fuel 22 serves as the heat sink gas 30. For example, in embodiments where the fuel 22 is essentially 100 mass percent hydrogen and the oxygen supply 26 is essentially 100 mass percent oxygen, the stoichiometric oxygen to fuel mass ratio is about 8/1. Adding excess hydrogen such that the fuel 22 is 100% in excess of the stoichiometric oxygen to fuel mass ratio (where the oxygen to fuel mass ratio is 4/1) results in a nozzle exit temperature of about 1,600° C. at Mach 3. Fuel 22 can be added at a wide variety of percentages in excess of the stoichiometric oxygen to fuel mass ratio in various embodiments. To illustrate, when using essentially 100% hydrogen as the fuel 22 and 100% oxygen as the oxygen supply 26, the nozzle exit temperature at Mach 3 for 5% excess hydrogen (above the stoichiometric oxygen to fuel mass ratio) is about 2,305° C.; the nozzle exit temperature for 20% excess hydrogen is about 2,204° C., and the nozzle exit temperature for 200% excess hydrogen is about 1,118° C. Excess fuel 22 can be added at many different percentages above the stoichiometric oxygen to fuel mass ratio, and the amount of excess fuel 22 may depend on the fuel used, other temperature control steps, the desired nozzle exit temperature, and other factors. Other temperature control steps include adding a heat sink gas 30 other than fuel 22 in combination with adding excess fuel 22, or increasing the Mach number of the carrier gas at the exit of the converging/diverging nozzle 14.

The fuel 22 may be a mixture of different compounds, such as methane and hydrogen, and the amount of excess fuel above the stoichiometric oxygen to fuel mass ration may vary depending on the fuel 30 used. Hydrogen is relatively expensive, but methane produces carbon dioxide, and may produce carbon monoxide and/or soot, so the fuel 22 may be a mixture of hydrogen and methane. The fuel 22 may also include other components. In various exemplary embodiments, the fuel 22 may be about 99 or 100 percent mixed hydrogen and methane, where the hydrogen is present at about 0 mass percent, about 10 mass percent, about 25 mass percent, about 40 mass percent, about 50 mass percent, about 75 mass percent, about 90 mass percent, about 100 mass percent, or essentially any other percentage.

The table below lists example temperatures and mass flow rates for the reactor 10 based on mathematical models (not actual test results). In the examples, the fuel 22 is about 99 mass percent or more hydrogen, the oxygen supply 26 is about 99 mass percent or greater oxygen, the heat sink gas 30 is steam, and flow rates are expressed as kilograms per hour (kg/hr). The heat sink gas 30 is assumed to completely mix and equilibrate with the carrier gas. In all the examples, the methane supply 40 provided is about 1,670 kilograms per hour of methane to the reactor 10. The calculations for the examples were performed assuming equilibrium conditions in the converging/diverging nozzle 14.

### Example Nozzle Exit Temperature Controls

| | Example 1 | Example 2 | Example 3 | Example 4 |
|---|---|---|---|---|
| Fuel temp (° C.) | 800 | 800 | 800 | 800 |
| Fuel flow (kg/hr) | 202 | 202 | 400 | 320 |
| Oxygen temp (° C.) | 25 | 25 | 25 | 25 |
| Oxygen flow (kg/hr) | 1600 | 1600 | 1600 | 1600 |
| Steam temp (° C.) | N/A | 200 | N/A | 200 |
| Steam flow (kg/hr) | 0 | 950 | 0 | 500 |
| Mach number | 3.0 | 3.0 | 3.0 | 3.0 |
| Nozzle exit temp (° C.) | 2,307 | 1,815 | 1,598 | 1,651 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

1. A method of producing acetylene, the method comprising the steps of:
combusting a fuel with oxygen in a combustor to produce a carrier gas;
accelerating the carrier gas to a supersonic speed in a converging/diverging nozzle prior to the carrier gas entering a reaction zone;
controlling a nozzle exit temperature of the carrier gas from about 1,200° C. to about 2,500° C.;
adding methane to the carrier gas in the reaction zone; and
converting kinetic energy in the carrier gas to thermal energy in the reaction zone to increase the temperature of the carrier gas such that the methane reacts by pyrolysis to form the acetylene.

2. The method of claim 1 wherein controlling the nozzle exit temperature comprises adding a heat sink gas to the carrier gas before the reaction zone.

3. The method of claim 2 wherein the heat sink gas comprises one or more of steam, carbon dioxide, carbon monoxide, nitrogen, argon, or helium.

4. The method of claim 2 wherein the heat sink gas is the fuel and wherein the fuel is added to the combustor in excess of a stoichiometric oxygen to fuel ratio.

5. The method of claim 4 wherein the fuel is 5-200% in excess of the stoichiometric oxygen to fuel ratio.

6. The method of claim 2 wherein the heat sink gas is preheated prior to mixing with the carrier gas.

7. The method claim 6 wherein the heat sink gas is superheated steam.

8. The method of claim 2 wherein the heat sink gas is added to the combustor.

9. The method of claim 2 wherein the heat sink gas is added between the combustor and the reaction zone.

10. The method of claim 2 wherein one or more of the fuel, the oxygen or the heat sink gas are preheated.

11. The method of claim 1 wherein one or more of the fuel or the oxygen are preheated before combusting the fuel with the oxygen.

12. The method of claim 1 wherein about 10 mass percent or more of the fuel comprises methane.

13. The method of claim 1 wherein the fuel comprises about 25 mass percent or more hydrogen.

14. The method of claim 1 wherein accelerating the carrier gas to the supersonic speed comprises accelerating the carrier gas to the supersonic speed of from about Mach 2 to about Mach 4.

15. The method of claim 1 further comprising:
lowering a temperature of a stream exiting the reaction zone with a quench fluid.

16. The method of claim 1 wherein the nozzle exit temperature of the carrier gas is from about 1,500° C. to about 1,900° C. as the carrier gas enters the reaction zone.

17. A reactor for producing acetylene comprising:
a combustor comprising a fuel inlet and an oxygen supply inlet;
a converging/diverging nozzle fluidly coupled to the combustor, wherein the converging/diverging nozzle is configured to accelerate a carrier gas to supersonic speeds;
a reaction zone fluidly coupled to the converging/diverging nozzle, wherein the reaction zone further comprises a methane inlet; and
a nozzle heat sink gas inlet in the reactor between the combustor and the reaction zone.

18. The reactor of claim 17 further comprising a fuel line in fluidly coupled with the fuel inlet, an oxygen supply line fluidly coupled with the oxygen supply inlet, and a pre-heater thermally coupled with one or more of the fuel line, the oxygen supply line, or heat sink gas line.

19. The reactor of claim 17 further comprising a heat sink gas inlet fluidly coupled to the combustor.

20. The reactor of claim 17 wherein the converging/diverging nozzle comprises a converging section fluidly coupled to a diverging section, the converging section having an inlet and an outlet and wherein a cross sectional area of the converging section decreases from the inlet to the outlet, and the diverging section having an inlet and an outlet and wherein a cross sectional area of the diverging section increases from the inlet to the outlet.
